(19) United States
(12) Patent Application Publication
(10) Pub. No.: US 2021/0361897 A1
(43) Pub. Date: Nov. 25, 2021

(54) APPARATUS AND METHOD FOR SELECTING POSITIVE AIRWAY PRESSURE MASK INTERFACE

(71) Applicants: Lawrence Neal, Portland, OR (US); Sudesh Banskota, Hillsboro, OR (US); Akhil Raghuram, Vancouver, WA (US)

(72) Inventors: Lawrence Neal, Portland, OR (US); Sudesh Banskota, Hillsboro, OR (US); Akhil Raghuram, Vancouver, WA (US)

(21) Appl. No.: 17/242,600

(22) Filed: Apr. 28, 2021

Related U.S. Application Data
(60) Provisional application No. 63/028,351, filed on May 21, 2020.

(51) Int. Cl. A61M 16/06; G06K 9/00

(52) U.S. Cl. CPC A61M 16/0605; A61M 2205/3553; G06K 9/00288; G06K 9/00248

(57) ABSTRACT

Embodiments of the positive airway pressure (PAP) mask fitting system and method provide a PAP mask fitting process to a specific patient that is as automatic as possible and that returns the patient the most appropriate PAP mask fit. The PAP mask fitting is done in a relatively quick manner. Once a PAP mask fitting has identified a preferred PAP mask for the patient, an appropriate PAP mask can be ordered on demand and is quickly, and possibly immediately, provided to the patient.

### PRIORITY CLAIM

This application claims priority to copending U.S. Provisional Application, Ser. No. 63/028,351, filed on May 21, 2020, entitled Systems and Methods For Selecting Positive Airway Pressure Mask Interface, which is hereby incorporated by reference in its entirety for all purposes.

### BACKGROUND OF THE INVENTION

Obstructive and central sleep apnea are highly prevalent problems. Devices that provide positive airway pressure (PAP) are the treatment of choice for these patients. Such PAP devices are also interchangeably referred to continuous positive airway pressure (CPAP) devices in the arts. A PAP system entails the patient wearing a mask interface to deliver pressurized air to act as a pressure splint to keep their breathing airway open while they sleep. Patients still have to consider numerous PAP mask options available to find a compromise between fit, style, color, shape, price and so on.

Masks for PAP use are mass produced in standardized sizes. Each patient's face is sufficiently unique as a basic form of identification, but the patient has to choose from products made for general faces that differ from person to person. It is very difficult for the patient to find out a person's unique taste, facial skeleton and one perfect mask to suit their needs. Appropriate fit of the mask has been an ongoing challenge which is a barrier to appropriate treatment of these patients. The traditional approach and model of care has been for patients to visit a Home Medical Equipment (HME) office and have an expert fit the patient with an appropriate mask. This is a high cost method and the results of fitting the patient for their PAP mask during an in-person visit are clinician dependent and prone to variability.

Recent entrants attempting to perform remote mask fittings in lieu of in-person mask fittings have looked at having a patient use standard, easily available objects such as a US Quarter Dollar coin or ruler to measure their face and determine an appropriate mask. These fittings have been performed by means of a web-based teleconferencing software (Zoom/Skype) with an expert guiding the patient, or by the patient using a web based application guiding them through the steps. These are difficult to follow and have poor reliability.

Accordingly, in the arts of PAP system, and in particular PAP masks, there is a need for an improved process to fit a user with a PAP mask that best stats the user's needs and unique facial attributes.

### SUMMARY OF THE INVENTION

Embodiments of the positive airway pressure (PAP) mask fitting system and method provide a PAP mask fitting process to a specific patient that is as automatic as possible and that returns the patient the most appropriate PAP mask fit. The PAP mask fitting is done in a relatively quick manner. Once a PAP mask fitting has identified a preferred PAP mask for the patient, an appropriate PAP mask can be ordered on demand and is quickly, and possibly immediately, provided to the patient.

Further, with the ongoing COVID19 pandemic, in-office mask fitting is deemed a risky procedure to both patients (user) and clinicians. The ability for patients to get fit with an appropriate mask at home using their smartphone or other image capture device is facilitated using embodiments of the PAP mask fitting system and method.

### BRIEF DESCRIPTION OF THE DRAWINGS

The components in the drawings are not necessarily to scale relative to each other. Like reference numerals designate corresponding parts throughout the several views.

FIG. 1 is a combination flow diagram and block diagram of a PAP mask fitting system.

FIGS. 2 and 3 are conceptual diagrams illustrating location and vectors of selected face key points.

FIG. 3A-3C are operational schematics of PAP mask fitting system and method mask selection tool data flow between a patient's mobile device and a remote clinic dashboard.

FIG. 4 is a block diagram showing additional detail of an example PAP mask fitting system.

### DETAILED DESCRIPTION

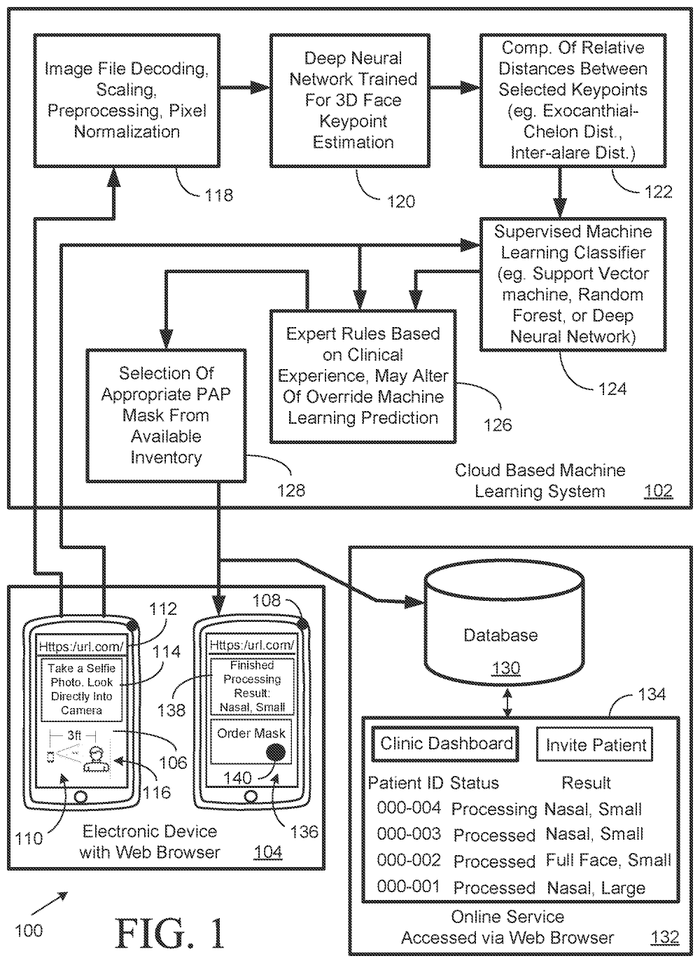

FIG. 1 illustrates an example positive airway pressure (PAP) mask fitting system 100. Embodiments of the PAP mask fitting system 100 provide a system and method for identifying selected face key points from a received image of the patient's face who is to be fitted for a PAP mask. Based upon characteristics of and relationships between the identified face key points, a particular PAP mask may be identified for a PAP patient.

The disclosed systems and methods for a PAP mask fitting system 100 will become better understood through review of the following detailed description in conjunction with the figures. The detailed description and figures provide examples of the various inventions described herein. Those skilled in the art will understand that the disclosed examples may be varied, modified, and altered without departing from the scope of the inventions described herein. Many variations are contemplated for different applications and design considerations, however, for the sake of brevity, each and every contemplated variation is not individually described in the following detailed description.

Throughout the following detailed description, a variety of examples for systems and methods for the PAP mask fitting system 100 are provided. Related features in the examples may be identical, similar, or dissimilar in different examples. For the sake of brevity, related features will not be redundantly explained in each example. Instead, the use of related feature names will cue the reader that the feature with a related feature name may be similar to the related feature in an example explained previously. Features specific to a given example will be described in that particular example. The reader should understand that a given feature need not be the same or similar to the specific portrayal of a related feature in any given figure or example.

The following definitions apply herein, unless otherwise indicated.

“Substantially” means to be more-or-less conforming to the particular dimension, range, shape, concept, or other aspect modified by the term, such that a feature or component need not conform exactly. For example, a “substantially cylindrical” object means that the object resembles a cylinder, but may have one or more deviations from a true cylinder.

“Comprising,” “including,” and “having” (and conjugations thereof) are used interchangeably to mean including but not necessarily limited to, and are open-ended terms not intended to exclude additional, elements or method steps not expressly recited.

Terms such as “first”, “second”, and “third” are used to distinguish or identify various members of a group, or the like, and are not intended to denote a serial, chronological, or numerical limitation.

“Coupled” means connected, either permanently or releasably, whether directly or indirectly through intervening components.

“Communicatively coupled” means that an electronic device exchanges information with another electronic device, either wirelessly or with a wire based connector, whether directly or indirectly through a communication network 108. “Controllably coupled” means that an electronic device controls operation of another electronic device.

Returning to FIG. 1, a combination flow diagram and block diagram of a PAP mask fitting system 100 is illustrated. A non-limiting embodiment of the PAP mask fitting system 100 employs a cloud based machine learning system 102 that receives image data from an electronic device 104 provisioned with a web browser and an image capture device 106.

In the hypothetical embodiment illustrated in FIG. 1, the electronic device 104 is generically illustrated as a smart phone provisioned with a display 106 and an image capture device 108 that is oriented inward so as to be configured to capture an image of the patient. The captured image of the patient is interchangeably referred to as a “selfie” herein.

Other types of electronic devices 104 may be used with embodiments of the PAP mask fitting system 100. For example, a laptop or personal computer provisioned with an image capture device (camera) may be used with the PAP mask fitting system 100. Other examples electronic devices 104 include cellular phones, notebooks, personal device assistants, or the like. The patient might even take a selfie with a legacy camera, and then email the captured image to the PAP mask fitting system 100. Any electronic device now known or later developed is intended to be within the scope of this disclosure.

To initiate operation of the PAP mask fitting system 100, the patient using their electronic device 104 initiates an interactive session with the cloud based machine learning system 102. Using a web interface, a clinical operator and/or the cloud based machine learning system 102 creates an electronic record specifying an individual patient. This record may include the patient's name, phone number, and medical information. In a non-limiting example embodiment, when the patient record is created, an SMS text message is automatically sent to the patient's mobile phone number. This text message contains an individualized message and a hyperlink 112, preferably to be opened one time only, by the patient, on their electronic device 104, such as their mobile smart phone device. The hyperlink address 112 is for a particular web site that is the portal for an interactive PAP mask fitting session. Alternatively, or additionally, the individualized message and the hyperlink 112 may be communicated to another designated electronic device.

After receiving the individualized message and the hyperlink 112, the patient logs in to a secure portal (server) of the cloud based machine learning system 102 to establish a secure interactive PAP mask fitting session. If the patient is using their smart phone 104, the patient may log in using, the SMS message text. The hyperlink directs the patient to a user interface, containing personalized messaging for this patient, a set of medical questions, and an optional photo upload button (that is later used to upload a capture image of the patient's face to the cloud based machine learning system 102). The user patient answers the medical questions through the Web interface via a presented graphical user interface (GUI). Example medical questions include, but are not limited to, sleep difficulties, breathing difficulties, preferences about wearing glasses, facial hair, dental problems, and other issues that may affect the proper choice of PAP equipment.

The patient is instructed to take a photograph of their own face using their mobile device 104 or another image capture device, according to some simple instructions such as, but not limited to, “Hold the camera at arm's length, hold the camera at eye level, look directly at the camera, and take the photo.” For example, the electronic device 104 receives a GUI 110 that is presented on the display 106. The non-limiting GUI 110 presents information indicating the hyperlink address 112, textual user instructions 114 for capturing a selfie image, and/or a graphical image 116 that graphically instructs the patient. Based on the instructions, the patient captures an image of their face. Any suitable GUI, or series of GUIs, may be used to facilitate the capture of an image of the patient's face. Other alternative GUIs may present more information, less information, and/or different information to guide the patient through the interactive PAP mask fitting session.

Once the image of the patient has been acquired, the image data is communicated to the cloud based machine learning system 102. Preferably, only a single image of the patient's face is required. Alternatively, or additionally, multiple images of the patient's face may be acquired. Multiple images may be taken from different angles of the patient's face, such as a side view or the like. In some embodiments, the patient may capture a short video clip of their face from which multiple 2D images may be acquired from. In some embodiments, the video may be live streamed to the PAP mask fitting system 100 for a real time, or a near real time, PAP mask fitting process.

Various supplemental information may also be input by the patient via the presented GUIs. For example, the patient may input their name, age, sex, contact information, health provider information, location information, account information, or the like that will be used to facilitate procuring a PAP mask for the patient.

The communicated image data of the patient's face is received and decoded at block 118. The image data of the patient's face is converted to an uncompressed format, scaled, and/or cropped to the appropriate size, and normalized to a format appropriate for input to a convolutional neural network. For example, but not limited to, the image data is processed by scaling the image of the patient's face to a standard size. In some embodiments, pixel normalization may be conducted so that the pixels of the preprocessed image data corresponds to the pixel attributes of a normalized face image. Some embodiments may adjust pixel brightness, luminosity, granularity, and/or color of the received image pixel data. The pixel data may be adjusted using any suitable algorithm now known or later developed.

In a preferred embodiment, at block 120, the pixel array (the processed image data) is fed as input to a trained convolutional neural network which predicts 3D positions of face key points from 2D image data. In a preferred embodiment, the convolutional neural network is a deep neural network. Any suitable convolutional neural network now known or later developed may be used in the various embodiments.

The deep neural network is trained to recognize two dimensional (2D) key face points of the patient's face in the 2D processed image data. The deep neural network determines corresponding three dimensional (3D) face key points. In three dimensions, the determined 3D face key points are defined in 3D space with respect to a reference point. Here, the neural network has already been trained using a large representative dataset of human faces, not necessarily limited to PAP patients. Any suitable neural network or suitable algorithm now known or later developed that identifies the face key points in the received 2D image data of the patient's face to determine corresponding 3D face key points may be used in alternative embodiments.

The neural network outputs a set of 3D points in a pre-specified order, corresponding to the estimated spatial location of face landmark points. The points include the corners of the mouth (Chelion left and right), corners of the inside of the eyes (Endocanthion left and right), corners of the outside of the eyes (Exocanthion), outer edges of the nose (Alare), bridge of the nose (Nasion) and other key face points. Some embodiments may identify boundaries of the eye iris for each eye. The iris data may be used to, but is not limited to, defining a scale factor of the patient's head. Any suitable number of and/or types of face key points may be determined in 3D space by the various embodiments.

There are various key point identification modules that perform this task, usually used for face recognition, emotion detection, or safety tasks. Some non-limiting examples include:
a A Face Alignment Network method.
b. A Joint Face Alignment and 3D Face Reconstruction.
c. A faster than real-time facial alignment such as 3d spatial transformer network approach in unconstrained poses.
d. A Pose-Invariant 3D Face Alignment method.
e. A “pose-invariant face alignment method.
f. Other types of convolutional networks, including generative adversarial networks, recurrent neural networks, and non-convolutional methods.

At block 122, a set of Euclidean distances between face key points are calculated, including the inter-alare distance (width of the nose), the chelion-to-exocanthion distance (height of the face from lips to eyes), and other relevant distances between face key points. Since the location information for each of the identified face key points is defined in 3D space, the computed distances may be represented as vectors in 3D space by some embodiments. Any suitable 3D coordinate system may be used by the various embodiments to compute these distances. Further, angles associated with each computed distance are determined to generate a vector.

At block 124, these vectors, along with the patient's answers to the web interface questions, and the medical information in the record created by the clinical operator and/or the cloud based machine learning system 102, are converted to a format appropriate for input to a supervised machine learning classifier. For example, the inputs may be converted to floating point numbers, and statistically standardized (subtracted from a predetermined mean value and divided by a predetermined standard deviation). The converted inputs are referred to as a facial feature vector.

The facial feature vector is input to a supervised machine learning classifier, which has already been trained with a pre-existing reference dataset, for the task of mask type classification. The classifier may be a Support Vector Machine, Random Forest, Logistic Regression, Deep Neural Network, or other employ another similar method. In some embodiments, the classifier may be an ensemble: a set of multiple SVM, Random Forest, etc., or a combination of such, each of which outputs an independent prediction, and whose predictions are averaged or otherwise aggregated to produce a final prediction. Each classifier predicts, given an input feature vector, which one out of a set of known CPAP mask types (full face, nasal, nasal pillows, etc.) is most likely to correctly fit the patient described by the feature vector. In some embodiments, Multi-Label Classification may be used to account for the possibility that more than one mask type may be appropriate for the patient, the supervised classifier may generate multiple predictions, one for each mask type, indicating the probability of fit. Then, a mask type and/or size prediction is computed. In addition to mask type, another classifier of the same type may be used to predict mask size, out of the set of possible sizes (small, medium, large, etc.). This size classifier may be independent of the type classifier, in which case the size and type are each predicted independently from the same facial feature vector, or the two classifiers may be integrated together, in which case a single prediction is made (e.g. Medium Nasal, Small Full-Face, etc.)

In some embodiments, demographics of the patient may be incorporated into the PAP mask fitting process. In such embodiments, members of a particular demographic category may have one or more facial and/or medical attributes that are relatively common along their demographic. Demographics may include age, sex, race, or the like of the patient undergoing the PAP mask fitting process.

At block 126, the prediction output by the supervised classifier, along with the demographic and medical information collected from the patient and input by the operator, is input to a software component that applies a set of predetermined rules based on clinical knowledge, which may augment or override the machine learning output. For example, but not limited to, if a patient has claustrophobia, then the PAP mask fitting system 100 would not recommend a full-face style mask. Given the recommended PAP mask type/size (e.g. Nasal Mask, Medium), and based on operator preferences and availability of supplies, one or more specific models of CPAP mask (e.g. Fisher & Paykel Eson 2 with Medium headgear and Medium cushion) are identified and are output at block 128.

The information identifying the recommended PAP model, after all rules have been applied, is stored in a database 130. In an example embodiment, the database 130 may reside at an online service accessed web browser 132. The PAP mask recommendations may be returned, via the Web interface 132, to both to the patient's electronic device 104 and to the clinical operator. The example data 134 may be stored in a relational database or the like that associates the user patient's identity, their processing status, and the resultant PAP mask recommendation.

In an example embodiment, a non-limiting example GUI 136 may then be presented to the patient on the display 116 of their electronic device 104. Textual information 138 indicating the PAP mask recommendation may be presented to the patient. Additionally, or alternatively, images of the recommended PAP mask (not shown) may be presented to the patient. Optionally, an active hot spot 140 on the touch sensitive display 116 of the patient's electronic device 104 may be provided to enable the patient to procure the recommended PAP mask.

Afterwards, additional follow-up communication may be sent to the patient or operator, to determine whether the recommended PAP mask was correct. This feedback information may be communicated back to the cloud based machine learning system 102 to enhance the learning of the neural network.

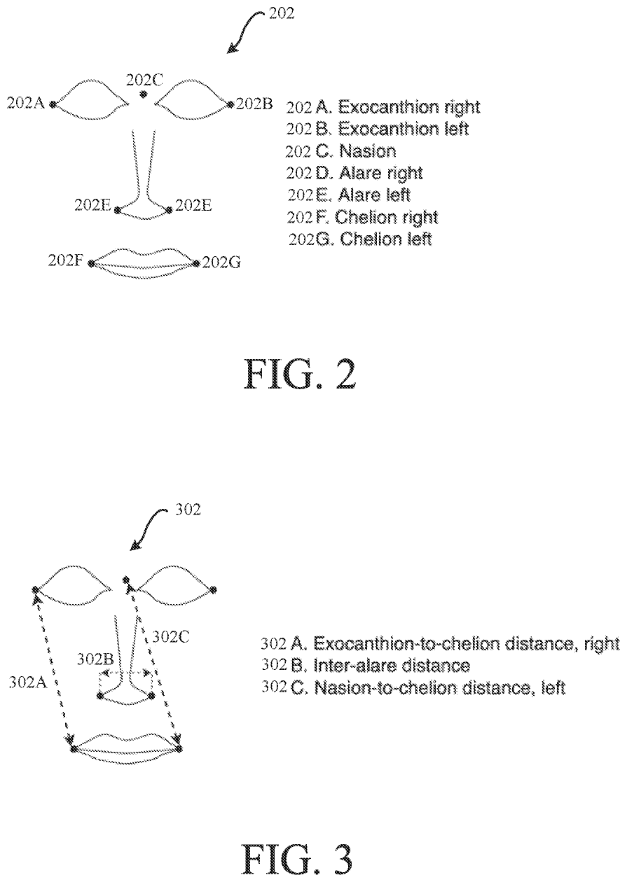

FIGS. 2 and 3 are conceptual diagrams illustrating location 202 and vector 302 between selected face key points. In an example embodiments, a generic human face is illustrated which shows various key face points that are determinable for a received image of the patient's face. Facial key points 202a and 202h correspond to the exocanthion right and the exocanthion left key face points, respectively. Facial key point 202c is a nasion face key point. Facial key points 202d and 202e are the alare right and alare left face key points, respectively. Facial key points 202f and 202g are the chelion right and chelion left face key points, respectively.

In FIG. 2, several example vectors that are computed during the PAP mask fitting process are illustrated. Vector 302a corresponds to the exocanthion-to cehlion distance vector, right. Vector 302b corresponds to the inter-alare distance vector. Vector 302c corresponds to the nasion-to-chelion distance vector, left. One skilled in the art appreciates that numerous additional vectors between selected face key points are determined during the PAP mask fitting process.

When a plurality of 2D images are used to determine the face key points in 3D space, location information of like face key points may be combined, averaged otherwise combined to improve the accuracy of the determined location of the patient's face key points.

When the distances and angles (vectors) between patient's face key points have been determined by the PAP mask fitting system 100, a patient's facial feature vector is determined for the patient. In an example embodiment, the patient's facial feature vector may be represented in a matrix or other suitable format.

Each PAP mask has a corresponding facial feature vector. When a patient's facial feature vector matches or corresponds with the PAP mask facial feature vector of a particular PAP mask, that PAP mask may be identified as a suitable candidate PAP mask for consideration for use by the patient. It is likely that for any particular patient, a plurality of different PAP masks may be identified as candidate PAP masks.

Preferably, the distances and/or angles of the PAP mask facial feature vector are expressed in ranges. One skilled in the art appreciates that an exact match between a patient's facial feature vector and the PAP mask facial feature vector is at best problematic. However, when the distances and/or angles of the PAP mask facial feature vector are expressed as a range, then the probability of identifying a suitable candidate PAP mask increases to a point that it is highly likely that a suitable PAP mask may be identified for the patient.

Alternative embodiments may use other processes and/or systems for measuring a patient's face during implementation of a PAP mask fitting system 100. Various neural network types may be used in alternative embodiments without departing substantially from the scope of this disclosure, and are intended to be included herein as alternative embodiments protected by the claims herein.

Various embodiments may employ alternative, or additional, types of communication systems and analysis systems to allow clinicians and/or patients to send links to access various data and/or to receive results data. Such non-limiting features are intended to be included herein as alternative embodiments protected by the claims herein.

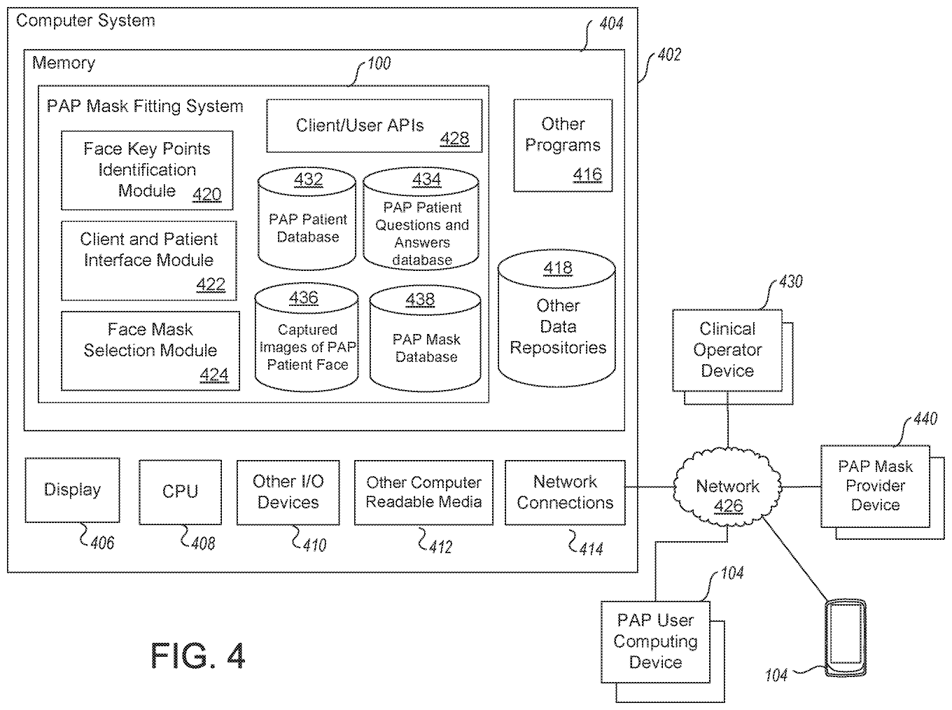

FIG. 4 is a block diagram showing additional detail of an example PAP mask fitting system implemented as an example computing system 402 that may be used to practice embodiments of PAP mask fitting system 100 described herein. Note that one or more general purpose virtual or physical computing systems suitably instructed or a special purpose computing system may be used to implement a PAP mask fitting system 100. Further, the PAP mask fitting system 100 may be implemented in software, hardware, firmware, or in some combination to achieve the capabilities described herein.

Note that one or more general purpose or special purpose computing systems/devices may be used to implement the described techniques. However, just because it is possible to implement the PAP mask fitting system 100 on a general purpose computing system does not mean that the techniques themselves or the operations required to implement the techniques are conventional or well known.

The computing system 402 may comprise one or more server and/or client computing systems and may span distributed locations. In addition, each block shown may represent one or more such blocks as appropriate to a specific embodiment or may be combined with other blocks. Moreover, the various blocks of the PAP mask fitting system 100 may physically reside on one or more machines, which use standard (e.g., TCP/IP) or proprietary interprocess communication mechanisms to communicate with each other.

In the embodiment shown, computer system 402 comprises a computer memory (“memory”) 404, a display 406, one or more Central Processing Units (“CPU”) 408, Input/Output devices 410 (e.g., keyboard, mouse, CRT or LCD display, etc.), other computer-readable media 412, and one or more network connections 414. The PAP mask fitting system 100 is shown residing in memory 404. In other embodiments, some portion of the contents, some of, or all of the components of the PAP mask fitting system 100 may be stored on and/or transmitted over the other computer-readable media 412. The components of the PAP mask fitting system 100 preferably execute on one or more CPUs 408 and manage the identification of candidate PAP masks based on the patient's facial feature vector determined from the image data of the patient's face, as described herein. Other code or programs 416 and potentially other data repositories, such as data repository 418, also reside in the memory 404, and preferably execute on one or more CPUs 408 to perform other tasks. Of note, one or more of the components in FIG. 4 may not be present in any specific implementation. For example, some embodiments embedded in other software may not provide means for user input or display.

In a typical embodiment, the PAP mask fitting system 100 includes one or more face key points identification module 420, a client and patient interface module 422, and a face mask selection module 424. In at least some embodiments, one of more of these modules 420, 422, 424 may be provided external to the computer system 402 and is available, potentially, over one or more networks 426.

The client and patient interface module 422 is configured to facilitate establishment of a communication link between the computer system 402, the patient's electronic device 104 and the clinical operator device. Information received about the patient is stored into the PAP patient database 432.

During the initial PAP mask fitting process, the patient is asked a series of medical health questions. The client and patient interface module 422 stored the questions and answers into the PAP patient questions and answers database 434.

The client and patient interface module 433 is also configured to facilitate receiving the 2D image of the patient's face. The client and patient interface module 422 then stores the received 2D image data into the captured images of PAP patient faces database 439.

The face key points identification module 420 is configured to determine the face key points and the resultant facial feature vector as described herein. A facial feature vector is a sequence of numbers that describe measurable properties of an object, wherein each vector is a mathematical representation of a direction and a magnitude (length). Alternative embodiments may employ any suitable form of expressing a facial feature vector now known or later developed. Once determined, the patient's face key points and the associated facial feature vector may be stored into the PAP patient database 432.

Information about the available PAP masks is received from the PAP mask provider device 440 in an example embodiment. In some embodiments, the face mask selection module 424 harvests information about the various available PAP masks and the associated PAP mask facial feature vectors from the various manufacturers and/or vendors of PAP masks. This information is stored in the PAP mask database 438.

Once the patients facial feature vector has been determined, the face mask selection module 424 compares the patient's unique facial feature vector with the PAP mask facial feature vectors for all of the available PAP masks. Those PAP masks have a PAP mask facial feature vector that corresponds with (or is compatible with) the patient's face key point are identified as candidate PAP masks.

As noted hereinabove, one of the later processes is to apply a set of predetermined rules based on clinical knowledge, which may augment, or potentially override, the machine learning output. The predetermined rules may be manually applied by a clinician. Alternatively, a neural module or other suitable module may apply the predetermined rules as part of the PAP mask fitting process being performed by the PAP mask fitting system 100. For example, but not limited to, if a patient has claustrophobia, then the PAP mask fitting system 100 would not recommend a full-face style mask. Given the recommended PAP mask type/size (e.g. Nasal Mask, Medium), and based on operator preferences and availability of supplies, one or more specific models of CPAP mask (e.g. Fisher & Paykel Eson 2 with Medium headgear and Medium cushion) are identified and are then output to the patient for their consideration.

In some embodiments, the supervised machine learning classifier may employ a classification system using a probabilistic graphical model module 442. The probabilistic graphical model module 442 may represent supervised machine learning output, hand-written clinical rules, inventory preferences, and anything else we add, are all represented as changes in probability, and can be merged together to produce one final answer.

Other and/or different modules may be implemented. In addition, the modules 420, 422, 424, 442 may interact via a network 426 with application or client code application program interfaces (APIs) 428 that facilitate communication with remote components, such as one or more clinical operator devices 430, such as purveyors of patient health and insurance account information stored in PAP Patient database 432. Also, of note, the PAP Patient database 432 may be provided external to the computer system 402 as well, for example in a WWW knowledge base accessible over one or more networks 426. In some embodiments, one or more of the modules 420, 422, 424, 442 may be merged together and/or merged with other modules.

In an example embodiment, components/modules of the PAP mask fitting system 100 are implemented using standard programming techniques. For example, the PAP mask fitting system 100 may be implemented as a “native” executable running on the CPU 103, along with one or more static or dynamic libraries. In other embodiments, the PAP mask fitting system 100 may be implemented as instructions processed by a virtual machine. In general, a range of programming languages known in the art may be employed for implementing such example embodiments, including representative implementations of various programming language paradigms, including but not limited to, object-oriented (e.g., Java, C++, C#, Visual Basic.NET, Smalltalk, and the like), functional (e.g., ML, Lisp, Scheme, and the like), procedural (e.g., C, Pascal, Ada, Modula, and the like), scripting (e.g., Perl, Ruby, Python, JavaScript, VBScript, and the like), and declarative (e.g., SQL, Prolog, and the like).

The embodiments described above may also use well-known or proprietary, synchronous or asynchronous client-server computing techniques. Also, the various components may be implemented using more monolithic programming techniques, for example, as an executable running on a single CPU computer system, or alternatively decomposed using a variety of structuring techniques known in the art, including hut not limited to, multiprogramming, multithreading, client-server, or peer-to-peer, running on one or more computer systems each having one or more CPUs. Some embodiments may execute concurrently and asynchronously and communicate using message passing techniques. Equivalent synchronous embodiments are also supported.

In addition, programming interfaces to the data stored as part of PAP mask fitting system 100 (e.g., in the data repositories 432, 434, 436, 438) can be available by standard mechanisms such as through C, C++, C#, and Java APIs; libraries for accessing files, databases, or other data repositories; through scripting languages such as XML; or through Web servers, FTP servers, or other types of servers providing access to stored data. The data repositories 432, 434, 436, 438 may be implemented as one or more database systems, file systems, or any other technique for storing such information, or any combination of the above, including implementations using distributed computing techniques.

Also the example PAP mask fitting system 100 may be implemented in a distributed environment comprising multiple, even heterogeneous, computer systems and networks. Different configurations and locations of programs and data are contemplated for use with techniques of described herein. In addition, the [server and/or client] may be physical or virtual computing systems and may reside on the same physical system. Also, one or more of the modules may themselves be distributed, pooled or otherwise grouped, such as for load balancing, reliability or security reasons. A variety of distributed computing techniques are appropriate for implementing the components of the illustrated embodiments in a distributed manner including but not limited to TCP/IP sockets, RPC, RMI, HTTP, Web Services (XML-RPC, JAX-RPC, SOAP, etc.) and the like. Other variations are possible. Also, other functionality could be provided by each component/module, or existing functionality could be distributed amongst the components/modules in different ways, yet still achieve the functions of a PAP mask fitting system 100.

Furthermore, in some embodiments, some or all of the components of the PAP mask fitting system 100 may be implemented or provided in other manners, such as at least partially in firmware and/or hardware, including, but not limited to one or more application-specific integrated circuits (ASICs), standard integrated circuits, controllers executing appropriate instructions, and including microcontrollers and/or embedded controllers, field-programmable gate arrays (FPGAs), complex programmable logic devices (CPLDs), and the like. Some or all of the system components and/or data structures may also be stored as contents (e.g., as executable or other machine-readable software instructions or structured data) on a computer-readable medium (e.g., a hard disk; memory; network; other computer-readable medium; or other portable media article to be read by an appropriate drive or via an appropriate connection, such as a DVD or flash memory device) to enable the computer-readable medium to execute or otherwise use or provide the contents to perform at least some of the described techniques. Some or all of the components and/or data structures may be stored on tangible, non-transitory storage mediums. Some or all of the system components and data structures may also be stored as data signals (e.g., by being encoded as part of a carrier wave or included as part of an analog or digital propagated signal) on a variety of computer-readable transmission mediums, which are then transmitted, including across wireless-based and wired/cable-based mediums, and may take a variety of forms (e.g., as part of a single or multiplexed analog signal, or as multiple discrete digital packets or frames). Such computer program products may also take other forms in other embodiments. Accordingly, embodiments of this disclosure may be practiced with other computer system configurations.

It should be emphasized that the above-described embodiments of the PAP mask fitting system 100 are merely possible examples of implementations of the invention. Many variations and modifications may be made to the above-described embodiments. All such modifications and variations are intended to be included herein within the scope of this disclosure and protected by the following claims.

Furthermore, the disclosure above encompasses multiple distinct inventions with independent utility. While each of these inventions has been disclosed in a particular form, the specific embodiments disclosed and illustrated above are not to be considered in a limiting sense as numerous variations are possible. The subject matter of the inventions includes all novel and non-obvious combinations and subcombinations of the various elements, features, functions and/or properties disclosed above and inherent to those skilled in the art pertaining to such inventions. Where the disclosure or subsequently filed claims recite “a” element, “a first” element, or any such equivalent term, the disclosure or claims should be understood to incorporate one or more such elements, neither requiring nor excluding two or more such elements.

Applicant(s) reserves the right to submit claims directed to combinations and subcombinations of the disclosed inventions that are believed to be novel and non-obvious. Inventions embodied in other combinations and subcombinations of features, functions, elements and/or properties may be claimed through amendment of those claims or presentation of new claims in the present application or in a related application. Such amended or new claims, whether they are directed to the same invention or a different invention and whether they are different, broader, narrower, or equal in scope to the original claims, are to be considered within the subject matter of the inventions described herein.

Therefore, having thus described the invention, at least the following is claimed:

1. A method of fitting a positive airway pressure (PAP) mask used by a patient, comprising:
receiving a two dimensional (2D) image of the PAP patient's face;
identifying a plurality of three dimensional (3D) face key points from the 2D image of the PAP patient;
computing a plurality of vectors between pairs of selected 3D face key points,
wherein the vector mathematically represents a distance and angle between paired of selected 3D face key points, and
wherein the plurality of vectors define a facial feature vector of the patient;
comparing the patient's facial feature vector with a corresponding plurality of predefined PAP mask face key point vectors for a plurality of available PAP masks; and
identifying a candidate PAP mask from the plurality of available PAP masks that has its corresponding PAP mask facial feature vector matches the determined patient's facial feature vector.

2. The method of claim 1, further comprising:
communicating a PAP mask recommendation to an electronic device of the PAP patient, wherein the PAP mask recommendation specifies at least a manufacturer of the candidate PAP mask and a size of the candidate PAP mask.

3. The method of claim 2, further comprising:
communicating with the PAP mask recommendation additional information indicating to the PAP patient where the recommended candidate PAP mask can be obtained.

4. The method of claim 2, wherein after communicating the PAP mask recommendation to the PAP patient, the method further comprising:
communicating a follow up questionnaire to the PAP patient, wherein the follow up questionnaire asks questions pertaining to the patient's satisfaction of the candidate PAP mask identified in the PAP mask recommendation; and
modifying the PAP mask recommendation based upon the received answers to the follow up questionnaire.

5. The method of claim 1, wherein prior to identifying the candidate PAP mask from the plurality of available PAP masks, the method further comprising:
communicating to the electronic device of the PAP patient a set of medical questions to be answered by the PAP patient:
receiving answers to the set of medical questions from the electronic device of the PAP patient; and
modifying identification of the candidate PAP mask based upon the received answers to the set of medical questions.

6. The method of claim 5, further comprising:
determining from the answers to the set of medical questions whether the PAP patient is claustrophobic; and
not identifying a full face PAP mask as the candidate PAP mask in response to determining that the PAP patient is claustrophobic.

7. The method of claim 1, wherein prior to receiving the 2D image, the method further comprising:
communicating instructions to an electronic device to the PAP patient,
wherein the communicated instructions specify procedures to the PAP patient pertaining to a capture of the 2D image of their face.

8. The method of claim 7, wherein communicating the instructions further comprises:
communicating a short message service (SMS) text message specifying the instructions to a cellular phone of the PAP patient,
wherein the capture 2D image is captured by the PAP patient with an image capture device on their cellular phone.

9. The method of claim 1, wherein after receiving the 2D image of the PAP patient, the method further comprising:
converting the image data of the 2D image of the PAP patient to an uncompressed image data format;
cropping the uncompressed image data so that an image of the PAP patient's face occupies a predefined amount of the total uncompressed image data; and
scaling the cropped uncompressed image data so that the face of the PAP patient is a predefined size that corresponds to standard facial size that fits each of the plurality of available PAP masks,
wherein the plurality of vectors between pairs of selected 3D face key points are computed from the scaled and cropped uncompressed image data.

10. The method of claim 9, further comprising:
aligning the image of the face of the PAP patient with a predefined standard alignment,
wherein the plurality of vectors between pairs of selected 3D face key points are computed based on the image data with the aligned face of the PAP patient.

11. The method of claim 1, wherein identifying the PAP mask from the plurality of available PAP masks comprises:
identifying a size of the candidate PAP mask.

12. The method of claim 1, wherein identifying the PAP mask from the plurality of available PAP masks comprises:
identifying a type of the candidate PAP mask from among the plurality of different types of PAP masks.

13. The method of claim 1, wherein the PAP patient is a first PAP patient, the method further comprising:
storing the plurality of vectors between pairs of selected 3D face key points into the database with an association with the first PAP patient, wherein information corresponding to a plurality of vectors between pairs of selected 3D face key points of the second PAP patient is compared to the plurality of vectors between pairs of selected 3D face key points of the first PAP patient in the learning process by a machine learning classifier;
comparing a candidate PAP mask for the second PAP patient with the candidate PAP mask identified for the first PAP patient; and
verifying the candidate PAP mask for the second PAP patient when plurality of vectors between pairs of selected 3D face key points is the same as the plurality of vectors between pairs of selected 3D face key points of the first PAP patient.

14. The method of claim 1, wherein the PAP patient is a first PAP patient, the method further comprising:
storing the plurality of identified 3D face key points in a database with an association with the first PAP patient;
comparing a plurality of identified 3D face key points identified in the 2D image of a second PAP patient to the stored plurality of identified 3D face key points of the first PAP patient in a learning process using a machine learning classifier, and
comparing a candidate PAP mask for the second PAP patient with the candidate PAP mask identified for the first PAP patient; and
verifying the candidate PAP mask for the second PAP patient when the candidate plurality of identified 3D face key points of the second PAP patient is the same as the identified 3D face key points of the first PAP patient.

15. The method of claim 1, wherein the received 2D image of the PAP patient is a first 2D image of the PAP patient, and wherein the plurality of vectors between pairs of selected 3D face key points are a first plurality of vectors between pairs of selected 3D face key points, the method further comprising:
receiving a second 2D image of the PAP patient's face;
identifying a second plurality of 3D face key points from the second 2D image of the PAP patient;
computing a second plurality of vectors between pairs of selected plurality of 3D face key points;
normalizing the second plurality of vectors between pairs of selected 3D face key points with the first plurality of vectors between pairs of selected 3D face key points determined from the first 2D image of the PAP patient; and
averaging each of the first and second plurality of vectors between pairs of selected 3D face key points to compute an average plurality of vectors between pairs of selected 3D face key points,
wherein the candidate PAP mask is identified based on the averaged plurality of vectors between pairs of selected 3D face key points.

16. The method of claim 15, wherein the first 2D image and the second 2D image of the PAP patient are in a video clip taken on the patient's face.

17. The method of claim 1, wherein the received 2D image of the PAP patient is a first 2D image of the PAP patient, and wherein the plurality of vectors between pairs of selected 3D face key points are a first plurality of vectors between pairs of selected 3D face key points, the method further comprising:
receiving a second 2D image of the PAP patient's face;
identifying a plurality of 3D face key points from the second 2D image of the PAP patient;
normalizing the second plurality of vectors between pairs of selected 3D face key points with the first plurality of identified 3D face key points determined from the first 2D image of the PAP patient;
comparing the second plurality of identified 31) face key points with the first plurality of identified 3D face key points; and
averaging each of the first and second plurality of plurality of identified 3D face key points to compute an average location for the plurality of identified 3D face key points,
wherein the plurality of vectors between pairs of selected 3D face key points are computed based on the averaged location for the plurality of identified 3D face key points.

18. The method of claim 1, further comprising:
receiving information about the plurality of available PAP masks from the makers of the plurality of available PAP masks, wherein the information specifies at least the plurality of vectors between pairs of selected 3D face key points for each one of the plurality of identified 3D face key points;
storing the received information about the plurality of available PAP masks in a database; and
accessing the stored information about the plurality of available PAP masks when the plurality of vectors between pairs of selected 3D face key points computed from the received 3D image of the PAP patient are compared with the corresponding plurality of PAP mask vectors for the plurality of available PAP masks.

19. The method of claim 1, further comprising:
receiving information about the plurality of available PAP masks from the makers of the plurality of available PAP masks;
computing the plurality of vectors between pairs of selected 3D face key points for each one of the plurality of available PAP masks based on the received information;
storing the computed plurality of vectors between pairs of selected 3D face key points in a database;
accessing the stored computed plurality of vectors between pairs of selected 3D face key points of the available PAP masks when the plurality of vectors between pairs of selected 3D face key points computed from the received 3D image of the PAP patient are compared with the corresponding plurality of PAP mask vectors for the plurality of available PAP masks.
